(19) United States
(12) Patent Application Publication
(10) Pub. No.: US 2025/0172523 A1
(43) Pub. Date: May 29, 2025

(54) RAPID, SINGLE-USE METHODS AND SYSTEMS FOR ELECTROCHEMICAL ANALYSIS OF PATHOGENS IN EXHALED BREATH

(71) Applicant: Washington University, St. Louis, MO (US)

(72) Inventors: Benjamin Sumlin, St. Louis, MO (US); Rajan Chakrabarty, St. Louis, MO (US); Nishit Jaideep Shetty, St. Louis, MO (US); John Cirrito, St. Louis, MO (US); Carla Yuede, St. Louis, MO (US)

(21) Appl. No.: 18/851,157

(22) PCT Filed: Mar. 30, 2023
(86) PCT No.: PCT/US2023/016822

§ 371 Date: Sep. 26, 2024

Related U.S. Application Data
(60) Provisional application No. 63/362,356, filed on Apr. 1, 2022.

(51) Int. Cl. G01N 27/327; A61B 5/097; G01N 33/543; G01N 33/569

(52) U.S. Cl. CPC G01N 27/3277; A61B 5/097; G01N 33/5438; G01N 33/56983; G01N 2333/165

(57) ABSTRACT

The present disclosure is directed to an exhaled breath condensate (EBC) collection device for collecting pathogens for analysis, the device comprising including: a breathing tube; and a condensing chamber located downstream from and in flow communication with the breathing tube, wherein where the condensing chamber includes an upper chamber and a lower chamber separated by a superhydrophobic surface.

### CROSS-REFERENCE TO RELATED APPLICATIONS

This application claims priority to U.S. Provisional Application Ser. No. 63/362,356, filed on Apr. 1, 2022, the content of which is hereby incorporated by reference in its entirety.

### STATEMENT REGARDING FEDERALLY SPONSORED RESEARCH DEVELOPMENT

This invention was made with government support under grant number U01AA029331 awarded by the National Institutes of Health. The government has certain rights in the invention.

### FIELD OF THE DISCLOSURE

The field of the disclosure relates generally to devices, systems, and methods for rapidly and reliably collecting and analyzing exhaled breath, and real-time detection of pathogens in exhaled breath.

### BACKGROUND OF THE DISCLOSURE

Coronavirus disease 2019 (COVID-19), first reported in December 2019, has swept the world, resulting in nearly 5.8 million deaths worldwide and more than 900,000 deaths in the United States as of early February 2022 (WHO website). The disease is caused by Severe Acute Respiratory Syndrome Coronavirus 2 (SARS-COV-2; CoV-2) which is primarily transmitted human-to-human through respiratory droplets and aerosols. COVID-19 vaccines have been effective in reducing hospitalizations and severe illness among vulnerable populations; however, the vaccines do not prevent human-to-human virus transmission and have reduced efficacy against new variants, such as omicron and BA.2. Current tests for CoV-2 vary in sensitivity, specificity, and test response times. Quantitative RT-PCR tests to detect viral RNA vary in response time from hours to days, while antibody tests generally take at least 1-2 days for results. Antigen tests tend to be the fastest, with current saliva or nasal swab results returned in as little as 5 minutes (e.g., Abbott COVID-19 ID NOW Test); however, these tests are invasive, have moderate specificity, and are not feasible to conduct rapidly on large groups of people. Moreover, the high rate of false positives with antigen tests can result in unnecessary and potentially hazardous therapeutic treatment of individuals who are not infected.

Rapid and real-time detection of the virus can help slow the spread by detecting the virus and allowing for quick disinfection, contact tracing and isolation. As the COVID-19 pandemic evolves and vaccination rates and efficacies remain insufficient to limit the spread of the disease, testing for variants is more important than ever.

One important lesson learned from SARS-COV-2 is the importance of limiting the transmission of emerging pathogens before widespread infection occurs. New tools that provide rapid results with high degree of specificity, and which can be developed and deployed rapidly in the field are needed to “get in front of” emerging pathogens that threaten to become the next epidemic and pandemic. This includes viruses, bacteria, parasites, fungi and mold. Moreover, known pathogens that are spread in enclosed spaces, such as hospitals, doctor's offices, long-term care facilities, and other healthcare settings can rapidly spread among vulnerable populations, resulting in nosocomial or hospital acquired infections (HAI), and new tools to rapidly diagnose these pathogens are necessary.

Accordingly, there is a need for rapid disposable, single-use, low-cost, detection techniques and tests that are envisioned for scalable production and use in hospitals, airports, schools, and anywhere where a large number of people are expected to gather. In particular, there is a need for electrochemical methods that offer improved limits of detection and higher fidelity than currently available “rapid” antigen tests. The embodiments described herein resolve at least these known deficiencies.

### BRIEF DESCRIPTION OF THE DISCLOSURE

In one aspect, the present disclosure is directed to an exhaled breath condensate (EBC) collection device for collecting pathogens for analysis, the device comprising: a breathing tube; and a condensing chamber located downstream from and in flow communication with the breathing tube, wherein the condensing chamber includes an upper chamber and a lower chamber separated by an inclined superhydrophobic surface.

In some embodiments, the EBC collection device further comprises a biosensor. In some embodiments, the inclined superhydrophobic surface is inclined at about 10 degrees to about 45 degrees, the superhydrophobic impaction surface is a polyimide film, the polyimide film is coated with a spray-coating of silicone wax, the lower chamber contains a cooling fluid, the cooling fluid is water, and/or the cooling fluid is produced from a user-initiated endothermic reaction. In some embodiments, the EBC device further comprises at least one transport fluid reservoir located upstream from and in flow communication with the condensing chamber. In some embodiments, the at least one transport fluid reservoir contains a buffer. In some embodiments, the buffer is phosphate-buffered saline. In some embodiments, the at least one transport fluid reservoir contains a blocking agent. In some embodiments, the blocking agent is bovine serum albumin. In other embodiments, the at least one transport fluid reservoir contains a combination of phosphate-buffered saline and bovine serum albumen. In some embodiments, the EBC device further comprises at least one disinfectant reservoir located upstream from and in flow communication with the condensing chamber. In some embodiments, the at least one disinfectant reservoir contains hypochlorous acid (HOCl). In some embodiments, the EBC device is disposable.

In another aspect, the present disclosure is directed to a method for detecting pathogens in exhaled breath, the method comprising: introducing an exhaled breath sample into an exhaled breath condensate (EBC) collection unit; condensing the exhaled breath sample into a liquid phase sample; contacting the liquid phase sample with a biosensor; measuring an output of the biosensor; and detecting at least one pathogen in the exhaled breath sample.

In some embodiments, the exhaled breath sample is condensed on a superhydrophobic impaction surface. In some embodiments, the method further comprises contacting the superhydrophobic impaction surface with a transport fluid to wash the condensed liquid phase sample onto the biosensor. In some embodiments, measuring the output of the biosensor comprises connecting a potentiostat unit to the biosensor and measuring a current output of the biosensor, wherein the current output of the biosensor is based on a square wave voltammetry measurement of tyrosine oxidation of tyrosine. In some embodiments, the method further comprises flushing the EBC with a disinfectant and disposing of the EBC unit.

In yet another aspect, the present disclosure is directed to a system for detecting pathogens, the system comprising: a disposable exhaled breath condensate (EBC) collection unit; and a portable potentiostat unit.

In some embodiments, the portable PU comprises a potentiostat, a battery, a user interface, and a microcontroller, and the portable PU is reusable.

In some embodiments, the system for detecting pathogens is used to detect viruses, bacteria, parasites, fungi, mold, or a combination thereof. In some embodiments, the system for detecting pathogens is used to detect multiple pathogens in a single test. In other embodiments, the system for detecting pathogens is used to detect multiple pathogens simultaneously in a single test, wherein the multiple pathogens are selected from combinations of viruses, bacteria, parasites, fungi, and mold, or multiple viruses, multiple bacteria, or multiple species or strains of viruses, bacteria, parasites, fungi, or mold. In some embodiments, the system for detecting pathogens is used to test for multiple variants of a pathogen (e.g., delta vs. omicron variants of SARS-COV-2) from a single test. In other embodiments, the system for detecting pathogens is used in multiplex tests in which it detects multiple pathogens simultaneously.

### BRIEF DESCRIPTION OF THE DRAWINGS

The embodiments described herein may be better understood by referring to the following description in conjunction with the accompanying drawings.

FIG. 1 is an exemplary embodiment of SARS-COV-2 biosensor design in accordance with the present disclosure. The CoV-2 biosensor uses square wave voltammetry to measure oxidation of tyrosine amino acids within the viral particle. Oxidation releases electrons that the sensor detects as current. The biosensor uses a nanobody attached to the surface to provide specificity and concentrate the viral particle at the electrode for measurement. The electrode is blocked with albumin to limit non-specific signal.

FIG. 2 is an exemplary embodiment of AB MIE biosensors used in the brains of living mice in accordance with the present disclosure. ISF AB40 concentrations over 180 minutes in APP/PS1 (n=6) and WT (n=3) mice.

FIGS. 3A-3D depict an exemplary embodiment of breathalyzer technology in accordance with the present disclosure. FIG. 3A shows a separated schematic of the EBC device. A user breathes into the tube. On the inside is a thermoconductive film (orange) covered in a hydrophobic layer that is cooled to 10° C. The exhaled breath is condensed on the surface then washed onto the biosensor using a transport media to measure for one or more pathogens. FIG. 3B shows a prototype design printed in resin. FIG. 3C shows an annotated interconnection between the EBC device and a portable, battery-powered electrometer. FIG. 3D shows a method for detecting pathogens in exhaled breath.

FIG. 4 is an exemplary embodiment of oxidation peak signals measured using the biosensor for varying concentrations of CoV-1 and CoV-2 spike protein on the same electrode in accordance with the present disclosure.

FIG. 5 is an exemplary embodiment of a dilution series of inactivated viral particles on the CoV-2 biosensor in accordance with the present disclosure. Chemically inactivated viral particles of the WA.1, beta, delta, and omicron (BA.1) strains were tested.

FIGS. 6A-6B depict an exemplary embodiment of aerosols with inactivated viral particles were nebulized, collected by the EBC, then detected on the biosensor in accordance with the present disclosure. FIG. 6A shows an example of the size distribution of the aerosolized particles, with a peak around 100 nm in diameter. FIG. 6B shows Iₒₓ current for aerosols collected in the EBC then readily detected by the biosensor. All viral samples tested (n=10) provided a positive signal above nebulized control solution.

FIGS. 7A-7D depict an exemplary embodiment of a central analysis unit in accordance with the present disclosure. FIG. 7A shows a front of the device. FIG. 7B shows an internal view of a microcontroller and peristaltic pumps. FIG. 7C shows a rear of the device, which has fluid ports, power entry, and an optional networking port. FIG. 7D shows an internal view, which has pumps, power supply, and a location for an electrode vial (not installed).

### DETAILED DESCRIPTION OF THE DISCLOSURE

The methods and systems described herein embody low-cost, rapid detection techniques in a fast, disposable, single-use test envisioned for scalable production (e.g., for use in hospitals, airports, schools, military bases, military vessels, and anywhere where a large number of people are expected to gather). Results of the rapid test methods and systems disclosed herein include real-time results as well as results in less than about 120s, about 90s, about 75s, about 60s, about 45s, about 30s, or about 15s. Further, sampling, sample processing, analyzing and/or detecting techniques may be carried out in a sterile environment (or environments), depending upon the embodiment. The electrochemical methods and systems exemplify improved limits of detection and higher fidelity than current available “rapid” antigen tests.

According to the present disclosure, significant progress has been made in the development of a breathalyzer for real-time detection of aerosolized pathogens (including SARS-COV-2) using an immuno-based biosensor. The breathalyzer is primarily for rapid testing of infected individuals (both symptomatic and asymptomatic) at a diagnostic level within 60 seconds of breathing into the device with the expectation that follow-up testing is not required. A single-use disposable exhaled breath condensate (EBC) collection device has been developed for capturing breath aerosols. The patient blows into the collection device which consists of a cooled (˜10° C.) hydrophobic film on the interior. The temperature difference between the exhaled breath and cool surface leads to condensation-based growth and collection of aerosol particles. Test buffer is added to collect the breath condensate which is applied to the biosensor for detection. In some embodiments, the breath condensate is applied to the biosensor and subsequently analyzed within several seconds or minutes of addition of the test buffer. In other embodiments, an exhaled breath sample may be collected (e.g., condensed and washed with test buffer) and suitably stored for a period of time (e.g., hours, days, weeks, etc.) prior to contact with the biosensor for pathogen detection. For instance, remote or at-home testing EBC collection devices may be provided such that collected samples require storage and/or transportation prior to contact with a biosensor for analysis. Depending on the embodiment, the breathalyzer is deployable to hospitals, schools, airports, and military facilities/vessels where a long queue of individuals needs to be rapidly tested. These devices will provide rapid readouts and act as a platform to detect other respiratory viruses and emerging pathogens.

Key performance metrics demonstrated for the devices described herein are their level of detection (LoD), specificity for target, particle collection efficiency, and longevity. During development, the sampled aerosols were subjected to different environmental conditions such as varying relative humidity, temperature, and contaminates (biological and environmental) to test their applicability in real-world scenarios. To manipulate and test environmental aerosols, a custom Goldberg drum was built to maintain aerosols at specific sizes over long periods of time with the ability to manipulate temperature, relative humidity, UV and add environmental contaminants (volatile organic compounds, primary organic compounds, ammonium sulfate, etc.).

Limit of detection (LoD)) of the biosensor: The biosensors were tested with varying concentrations of chemically-inactivated SARS-COV-2 viral particles. WA.1, beta, delta, and omicron BA.1 strains were tested with high sensitivities on the biosensor at 10-50 viral particles/virions/RNA copies per sample as verified by qRT-PCR. In some embodiments, the devices and systems disclosed herein have a limit of detection of less than 10 viral particles.

Specificity for target: In exemplary embodiments, the biosensor nanobody detects the repeat binding domain of the SARS-COV-2 spike protein. To determine specificity, the biosensor was tested against recombinant SARS-COV-2 compared to a comparable protein sequence in SARS-COV-1. CoV-1 produced negligible signal on the biosensor with over a 1,000-fold selectivity for CoV-2 over CoV-1 spike protein. In some embodiments, the biosensor detects inactivated viral particles of at least one respiratory virus (or several respiratory viruses) alternatively or additional to SARS-COV-2 virus particles. In other embodiments, the biosensor detects at least one bacterial genus or species. In yet other embodiments, the device detects at least one parasite, fungi, or mold genus or species.

Viral particles detectable by the disclosed biosensor include, but are not limited to, viruses associated with Chikungunya, Cholera, Crimean-Congo hemorrhagic fever, Ebola virus disease, Hendra virus infection, Influenza (pandemic, seasonal, zoonotic), Lassa fever, Marburg virus disease, Meningitis, MERS-COV, Monkeypox, Nipah virus infection, Novel coronavirus (2019-nCOV), Plague, Rift Valley fever, SARS, Smallpox, Tularaemia, Yellow fever, Zika virus disease, Ebola and Marburg virus (Filoviridae); Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichinde virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); influenza A virus (Orthomyxoviridae); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Ebola and Marburg virus (Filoviridae); Semliki Forest virus, Ross River virus, chikungunya virus, O'nyong-nyong virus, Sindbis virus, eastern/western/Venezuelan equine encephalitis virus (Togaviridae, Alphavirus); rubella (German measles) virus (Togaviridae, Rubivirus); rabies virus, Lagos bat virus, Mokola virus (Rhabdoviridae, Lyssavirus); Amapari virus, Pichinde virus, Tacaribe virus, Junin virus, Machupo virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); human immunodeficiency virus type 1 (Retroviridae, Lentivirus); influenza A/B virus (Orthomyxoviridae, the common ‘flu’ virus); respiratory syncytial virus (Paramyxoviridae, Pneumovirinae, Pneumovirus); Hendra virus, Nipah virus (Paramyxoviridae, Paramyxovirinae, Henipavirus); measles virus (Paramyxoviridae, Paramyxovirinae, Morbillivirus); Variola major (smallpox) virus (Poxviridae, Chordopoxvirinae, Orthopoxvirus); human hepatitis B virus (Hepadnaviridae, Orthohepadnavirus); hepatitis delta virus (hepatitis D virus) (unassigned Family, Deltavirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus), Adeno-associated virus Dependovirus, Parvoviridae Aichi virus Kobuvirus, Picornaviridae Australian bat lyssavirus, Rhabdoviridae BK polyomavirus, Polyomaviridae Banna virus Seadornavirus, Reoviridae Barmah forest virus Alphavirus, Togaviridae Bunyamwera virus Orthobunyavirus, Bunyaviridae Bunyavirus La Crosse Orthobunyavirus, Bunyaviridae Bunyavirus snowshoe hare Orthobunyavirus, Bunyaviridae Cercopithecine herpesvirus Lymphocryptovirus, Herpesviridae Chandipura virus Vesiculovirus, Rhabdoviridae Chikungunya virus Alphavirus, Togaviridae Cosavirus A Cosavirus, Picornaviridae Cowpox virus Orthopoxvirus, Poxviridae Coxsackievirus Enterovirus, Picornaviridae Crimean-Congo hemorrhagic Nairovirus, Bunyaviridae fever virus Dengue virus Flavivirus, Flaviviridae Dhori virus Thogotovirus, Orthomyxoviridae Dugbe virus Nairovirus, Bunyaviridae Duvenhage virus Lyssavirus, Rhabdoviridae Eastern equine encephalitis virus Alphavirus, Togaviridae Ebolavirus, Filoviridae Echovirus Enterovirus, Picornaviridae Encephalomyocarditis virus Cardiovirus, Picornaviridae Epstein-Barr virus Lymphocryptovirus, Herpesviridae European bat lyssavirus, Rhabdovirus GB virus C/Hepatitis G virus Pegivirus, Flaviviridae Hantaan virus Hantavirus, Bunyaviridae Hendra virus Henipavirus, paramyxoviridae Hepatitis A virus Hepatovirus, picornaviridae Hepatitis B virus Orthohepadnavirus, Hepadnaviridae Hepatitis C virus Hepacivirus, Flaviviridae Hepatitis E virus Hepevirus, Unassigned Hepatitis delta virus Deltavirus, Unassigned Horsepox virus Orthopoxvirus, Poxviridae Human adenovirus Mastadenovirus, Adenoviridae Human astrovirus Mamastrovirus, Astroviridae Human coronavirus Alphacoronavirus, Coronaviridae Human cytomegalovirus, Herpesviridae Human enterovirus 68, 70 Enterovirus, Picornaviridae Human herpesvirus 1 Simplexvirus, Herpesviridae Human herpesvirus 2 Simplexvirus, Herpesviridae Human herpesvirus 6 Roseolovirus, Herpesviridae Human herpesvirus 7 Roseolovirus, Herpesviridae Human herpesvirus 8 Rhadinovirus, Herpesviridae Human immunodeficiency virus Lentivirus, Retroviridae Human papillomavirus 1 Mupapillomavirus, Papillomaviridae Human papillomavirus 2 Alphapapillomavirus, Papillomaviridae Human papillomavirus 16, 18 Alphapapillomavirus, Papillomaviridae Human parainfluenza Respirovirus, Paramyxoviridae Human parvovirus B19 Erythrovirus, Parvoviridae Human respiratory syncytial virus Orthopneumovirus, Pneumoviridae Human rhinovirus Enterovirus, Picornaviridae Human SARS coronavirus Betacoronavirus, Coronaviridae Human spumaretrovirus Spumavirus, Retroviridae Human T-lymphotropic virus Deltaretrovirus, Retroviridae Human torovirus, Coronaviridae Influenza A virus Influenzavirus A, Orthomyxoviridae Influenza B virus Influenzavirus B, Orthomyxoviridae Influenza C virus Influenzavirus C, Orthomyxoviridae Isfahan virus Vesiculovirus, Rhabdoviridae JC polyomavirus, Polyomaviridae Japanese encephalitis virus Flavivirus, Flaviviridae Junin arenavirus, Arenaviridae KI Polyomavirus, Polyomaviridae Kunjin virus Flavivirus, Flaviviridae Lagos bat virus Lyssavirus, Rhabdoviridae Lake Victoria marburgvirus Marburgvirus, Filoviridae Langat virus Flavivirus, Flaviviridae Lassa virus Arenavirus, Arenaviridae Lordsdale virus Norovirus, Caliciviridae Louping ill virus Flavivirus, Flaviviridae Lymphocytic choriomeningitis Arenavirus, Arenaviridae virus Machupo virus Arenavirus, Arenaviridae Mayaro virus Alphavirus, Togaviridae MERS coronavirus Betacoronavirus, Coronaviridae Measles virus Morbilivirus, Paramyxoviridae Mengo encephalomyocarditis virus Cardiovirus, Picornaviridae Merkel cell polyomavirus, Polyomaviridae Mokola virus Lyssavirus, Rhabdoviridae Molluscum contagiosum virus Molluscipoxvirus, Poxviridae Monkeypox virus Orthopoxvirus, Poxviridae Mumps virus Rubulavirus, Paramyxoviridae Murray valley encephalitis virus Flavivirus, Flaviviridae New York virus Hantavirus, Bunyavirus Nipah virus Henipavirus, Paramyxoviridae Norwalk virus Norovirus, Caliciviridae O'nyong-nyong virus Alphavirus, Togaviridae Orf virus Parapoxvirus, Poxviridae Oropouche virus Orthobunyavirus, Bunyaviridae Pichinde virus Arenavirus, Arenaviridae Poliovirus Enterovirus, Picornaviridae Punta toro phlebovirus, Bunyaviridae Puumala virus Hantavirus, Bunyavirus Rabies virus Lyssavirus, Rhabdoviridae Rift valley fever virus Phlebovirus, Bunyaviridae Rosavirus A Rosavirus, Picornaviridae Ross river virus Alphavirus, Togaviridae Rotavirus A Rotavirus, Reoviridae Rotavirus B Rotavirus, Reoviridae Rotavirus C Rotavirus, Reoviridae Rubella virus Rubivirus, Togaviridae Sagiyama virus Alphavirus, Togaviridae Salivirus A Salivirus, Picornaviridae Sandfly fever sicilian virus Phlebovirus, Bunyaviridae Sapporo virus Sapovirus, Caliciviridae Semliki forest virus Alphavirus, Togaviridae Seoul virus Hantavirus, Bunyavirus Simian foamy virus Spumavirus, Retroviridae Simian virus 5 Rubulavirus, Paramyxoviridae Sindbis virus Alphavirus, Togaviridae Southampton virus Norovirus, Caliciviridae St. louis encephalitis virus Flavivirus, Flaviviridae Tick-borne powassan virus Flavivirus, Flaviviridae Torque teno virus Alphatorquevirus, Anelloviridae Toscana virus Phlebovirus, Bunyaviridae Uukuniemi virus Phlebovirus, Bunyaviridae Vaccinia virus Orthopoxvirus, Poxviridae Varicella-zoster virus Varicellovirus, Herpesviridae Variola virus Orthopoxvirus, Poxviridae Venezuelan equine encephalitis Alphavirus, Togaviridae virus Vesicular stomatitis virus Vesiculovirus, Rhabdoviridae Western equine encephalitis virus Alphavirus, Togaviridae WU polyomavirus, Polyomaviridae West Nile virus Flavivirus, Flaviviridae Yaba monkey tumor virus Orthopoxvirus, Poxviridae Yaba-like disease virus Orthopoxvirus, Poxviridae Yellow fever virus Flavivirus, Flaviviridae Zika virus Flavivirus, and Flaviviridae. Bacterial genera and species detectable by the disclosed biosensor include, but are not limited to, bacteria associated with Xanthomonas, Pseudomonas, Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Streptobacillus, Fusospirocheta, Spirillum, Ureaplasma, Spirochaeta, Mycoplasma, Actinomycetes, Borrelia, Bacteroides, Trichomoras, Branhamella, Pasteurella, Clostridium, Corynebacterium, Listeria, Bacillus, Erysipelothrix, Rhodococcus, Escherichia, Klebsiella, Pseudomanas, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia, Rochlimeae, as well as bacterial species such as: P. aeruginosa; E. coli, P. cepacia, S. epidermis, E. faecalis, S. pneumonias, S. aureus, N meningitidis, S. pyogenes, Pasteurella multocida, Treponema pallidum, and P. mirabilis. Gram-negative bacterial genera and species detectable by the disclosed biosensor include, but are not limited to, Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Citrobacter spp., Chlamydia spp., Brucella spp., Pseudomonas spp., Helicobacter spp. and Vibrio spp. Gram-negative bacterial genera and species detectable by the disclosed biosensor include, but are not limited to, Salmonella, E. coli, Yersinia pestis, Klebsiella and Shigella, Proteus, Enterobacter, Serratia, and Citrobacter.

Fungi detectable by the disclosed biosensor include, but are not limited to, fungi associated with Cryptococcus neoformans; Blastomyces dermatitidis; Aiellomyces dermatitidis; Histoplasma capsulatum; Coccidioides immitis; Candida species, including C. albicans, C. tropicalis, C. parapsilosis, C. guilliermondii and C. krusei, Aspergillus species, including A. fumigatus, A. flavus and A. niger, Rhizopus species; Rhizomucor species; Cunninghammella species; Apophysomyces species, including A. saksenaea, A. mucor and A. absidia; Sporothrix schenckii, Paracoccidioides brasiliensis; Pseudallescheria boydii, Torulopsis glabrata; Trichophyton species, Microsporum species and Dermatophyres species, as well as any other yeast or fungus now known or later identified to be pathogenic.

Parasites detectable by the disclosed biosensor include, but are not limited to, parasites associated with Anaplocephala, Ancylostoma, Necator, Ascaris, Brugia, Bunostomum, Capillaria, Chabertia, Cooperia, Cyathostomum, Cylicocyclus, Cylicodontophorus, Cylicostephanus, Craterostomum, Dictyocaulus, Dipetalonema, Dipylidium, Dracunculus, Echinococcus, Enterobius, Fasciola, Filaroides, Habronema, Haemonchus, Metastrongylus, Moniezia, Nematodirus, Nippostrongylus, Oesophagostomum, Onchocerca, Ostertagia, Oxyuris, Parascaris, Schistosoma, Strongylus, Taenia, Toxocara, Strongyloides, Toxascaris, Trichinella, Trichuris, Trichostrongylus, Triodontophorus, Uncinaria, Wuchereria, Leishmaniasis disease, human African trypanosomiasis disease, Chagas disease, antigens derived from members of the Apicomplexa phylum such as, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp.; Pneumocystis carinii; members of the Microspora phylum such as, for example, Nosema, Enterocytozoon, Encephalitozoon, Septata, Mrazekia, Amblyospora, Ameson, Glugea, Pleistophora and Microsporidium spp.; and members of the Ascetospora phylum such as, for example, Haplosporidium spp., as well as species including Plasmodium falciparum, P. vivax, P. ovale, P. malaria; Toxoplasma gondii; Leishmania mexicana, L. tropica, L. major, L. aethiopica, L. donovani, Trypanosoma cruzi, T brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T saginata, Trichomonas vaginitis, T hominis, T tenax; Giardia lamblia; Cryptosporidium parvum; Pneumocytis carinii, Babesia bovis, B. divergens, B. microti, Isospora belli, L. hominis; Dientamoeba fragilis; Onchocerca volvulus; Ascaris lumbricoides; Necator americans; Ancylostoma duodenale; Strongyloides stercoralis; Capillaria phihppinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phthirlus pubis; and Dermatobia hominis, as well as any other parasite now known or later identified to be pathogenic.

Additional pathogens detectable by the disclosed biosensor include, but are not limited to, Coronaviridae (e.g. MERS, SARS-COV-2), Bunyavirales (e.g. Lassa, Junin, Rift Valley Fever Virus, Andes, Sin Nombre, LaCrosse, California Encephalitis, Crimean Congo Hemorrhagic Fever), Filoviruses (e.g. Ebola, Marburg), Flaviviruses (e.g. Dengue, Zika, West Nile), Paramyxoviridae (e.g. Nipah, Hendra), Picornaviridae (e.g. EV-D68, EV-A71), Togaviridae (e.g. Chikungunya, EEE, VEE, WEE), Bacillus anthracis (including genotypic resistance markers), Yersinia pestis (including genotypic resistance markers), Francisella tularensis (including genotypic resistance markers), Burkholderia spp. (including genotypic resistance markers), Botulinum toxin (including identifying and distinguishing relevant serotypes), ESKAPE pathogens including genotypic resistance markers (e.g., Enterococcus faecium, Staphylococcus aureus, Klebsiella pneumonia, Acinetobacter baumannii, Pseudomonas aeruginosa, Enterobacter spp), Lassa virus, Nipah virus, Rift Valley Fever virus, Enterovirus D68 virus, Candida auris, Coccidioides sp., and novel coronaviruses.

Aspects of the present disclosure are provided by the subject matter of the following clauses:
1. An exhaled breath condensate (EBC) collection device for collecting pathogens for analysis, the device comprising:
   a breathing tube; and
   a condensing chamber located downstream from and in flow communication with the breathing tube, wherein the condensing chamber includes an upper chamber and a lower chamber separated by an inclined superhydrophobic impaction surface.
2. The EBC collection device of the preceding clause, wherein the EBC further comprises a biosensor.
3. The EBC collection device of any preceding clause, wherein the inclined superhydrophobic impaction surface is positioned at an incline of about 10 degrees to about 45 degrees.
4. The EBC collection device of any preceding clause, wherein the inclined superhydrophobic impaction surface comprises a hydrophobic surface.
5. The EBC collection device of any preceding clause, wherein the hydrophobic surface further comprises a coating.
6. The EBC collection device of any preceding clause, wherein the coating is a spray-coating.
7. The EBC collection device of any preceding clause, wherein the hydrophobic surface is a polyimide film and the spray-coating is silicone wax.
8. The EBC collection device of any preceding clause, wherein the lower chamber contains a cooling fluid.
9. The EBC collection device of any preceding clause, wherein the cooling fluid is water.
10. The EBC collection device of any preceding clause, wherein the cooling fluid is produced from a user-initiated endothermic reaction.
11. The EBC collection device of any preceding clause, further comprising at least one transport fluid reservoir located upstream from and in flow communication with the condensing chamber.
12. The EBC collection device of any preceding clause, wherein the at least one transport fluid reservoir contains at least one buffer.
13. The EBC collection device of any preceding clause, wherein the at least one buffer includes a phosphate buffer, a tris buffer, or a saline buffer.
14. The EBC collection device of any preceding clause, wherein the at least one transport fluid reservoir contains at least one blocking agent.
15. The EBC collection device of any preceding clause, wherein the at least one blocking agent includes at least one animal or plant based albumen.
16. The EBC collection device of any preceding clause, wherein the at least one transport fluid reservoir contains a combination of phosphate-buffered saline and bovine serum albumen.
17. The EBC collection device of any preceding clause, wherein the at least one transport fluid reservoir comprises a first transport fluid reservoir containing phosphate-buffered saline and a second transport fluid reservoir containing bovine serum albumen.
18. The EBC collection device of any preceding clause, further comprising at least one disinfectant reservoir located upstream from and in flow communication with the condensing chamber.
19. The EBC collection device of any preceding clause, wherein the at least one disinfectant reservoir contains hypochlorous acid (HOCI).
20. The EBC collection device of any preceding clause, wherein the EBC device is disposable.
21. A method for detecting pathogens in exhaled breath, the method comprising:
   introducing an exhaled breath sample into an exhaled breath condensate (EBC) collection unit;
   condensing the exhaled breath sample into a liquid phase sample;
   contacting the liquid phase sample with a biosensor;
   measuring an output of the biosensor; and
   detecting at least one pathogen in the exhaled breath sample.
22. The method of the preceding clause, wherein the exhaled breath sample is condensed on an inclined superhydrophobic impaction surface.
23. The method of any preceding clause, further comprising contacting the inclined superhydrophobic impaction surface with a transport fluid to wash the liquid phase sample onto the biosensor.
24. The method of any preceding clause, wherein measuring the output of the biosensor comprises connecting a potentiostat unit to the biosensor and measuring a current output of the biosensor.
25. The method of any preceding clause, wherein the current output of the biosensor is based on a square wave voltammetry measurement of tyrosine oxidation.
26. The method of any preceding clause, further comprising flushing the EBC collection unit with a disinfectant.
27. The method of any preceding clause, further comprising disposing of the EBC collection unit.
28. A system for detecting pathogens, the system comprising:
   a disposable exhaled breath condensate (EBC) collection unit; and
   a portable potentiostat unit.
29. The system of the preceding clause, wherein the system for detecting pathogens is used to detect viruses, bacteria, parasites, fungi, mold, or a combination thereof.
30. The system of any preceding clause, wherein the system for detecting pathogens is used to detect multiple pathogens in a single test.
31. The system of any preceding clause, wherein the system for detecting pathogens is used to detect multiple pathogens simultaneously in a single test, wherein the multiple pathogens are selected from combinations of viruses, bacteria, parasites, fungi, and mold, or multiple viruses, multiple bacteria, or multiple species or strains of viruses, bacteria, parasites, fungi, or mold.
32. The system of any preceding clause, wherein the system for detecting pathogens is used to test for multiple variants of a pathogen from a single test.
33. The system of any preceding clause, wherein the multiple variants of a pathogen include delta and omicron variants of SARS-COV-2.
34. The system of any preceding clause, wherein the system for detecting pathogens is used in multiplex tests, wherein multiple pathogens are detected simultaneously.

### EXAMPLES

Without further elaboration, it is believed that one skilled in the art using the preceding description can utilize the present disclosure to its fullest extent. The following Examples are, therefore, to be construed as merely illustrative, and not limiting of the disclosure in any way whatsoever.

Particle collection efficiency: The current EBC particle collection efficiency for the breathalyzer is 47.2±19.2%. This efficiency value was determined with aerosols nebulized at room temperature; however, aerosols in breath typically have a higher temperature and condense more readily on a cool surface which will increase collection efficiency. In some embodiments, the breathalyzer device has an aerosol collection efficiency above 70%.

Longevity: The biosensor was tested with repeated voltammetry cycles to determine the lifetime of the electrodes. Analyses shows that electrodes last for 70 sixty-second intervals before reduction in performance.

Cross-reactivity and interference from endogenous exogenous substances and microbes: The aerosol collection devices and the biosensor tests include those for cross-reactivity to other respiratory viruses and endogenous/exogenous substances found in breath as indicated by FDA guidance for the breathalyzer.

Proof-of-concept and prototypes for each part of the device were realized by the present disclosure, including SARS-COV-2 biosensors, potentiostat/electrometer/analysis unit, EBC collection device for the breathalyzer system and device. Overall, the disclosed solution entails the manufacture of a breathalyzer for point-of-care diagnosis of both symptomatic and asymptomatic pathogen detection (e.g., SARS-COV-2). As a testing aid, CHEST is an aerosol generation device which produces conditions and expelled particles analogous to those observed during respiratory exhalation and coughing. The length and interval of aerosol generation is user-controlled to mimic breathing patterns of individuals with different lung capacities and expiratory flow rates.

A nanobody-based electrochemical biosensor is used to selectively detect CoV-2 virions via the spike protein with a high sensitivity as low as 10-50 viral particles/sample. The technique provides instant results-within 60 seconds for the breathalyzer (for instance: 30 seconds to collect EBC, 15 seconds transfer to biosensor, 15 seconds to measure). The speed and sensitivity of this test enable improved prevention of indoor space disease transmission while simultaneously facilitating more indoor activity with a higher level of safety.

The described technology has been successful in detecting prevalent SARS-CoV-2 variants such as the WA1, B.1.351 (beta), B.1.617.2 (delta), and BA.1 (omicron) strains. Importantly the technology is a platform that is modifiable to detect other non-COVID pathogens such as influenza, OC43, MERS, and biological weapons such as anthrax. The breathalyzer device is herein enabled as a stand-alone instrument to be deployed to multiple locations and operated and maintained by technicians with minimal training.

Breathalyzer device testing according to the present disclosure involves using a device to mimic various types of expiration, including regular breaths, shallow breaths, talking, sneezing and coughing. As described herein, testing and optimization of each of the breathalyzer, environmental detector, and the biosensor includes interference from other factors, such as other respiratory viruses and endogenous/exogenous compounds in breath, as well as environmental pollutants.

As described herein, an electrochemical nanobody-based biosensor has been developed to detect aerosolized CoV-2 that will be deployed in a breathalyzer for real-time diagnosis. The environmental biosensor of the present disclosure for detecting target organisms is surprisingly and unexpectedly based on an ultra-sensitive electrochemical technology used in vivo (e.g., brain, tissue, interstitial fluid, etc.) for Alzheimer's disease research for detecting macromolecular targets.

The breathalyzer is deployable to testing locations that require rapid testing of a queue, such as lines to enter an immigration hall, a school, hospital, airplane, military vessel, or entertainment venue. In some embodiments, the breathalyzer device comprises a single-use disposable EBC collector (a cm3 box that the user blows into) that contains a tube for breathing into, a cold hydrophobic surface to condense the aerosols, separate chambers to hold wash buffer and HOCI disinfection solution, and the SARS-COV-2 biosensor. In some embodiments, the disposable EBC collector is then connected to a stationary, permanent analysis unit that contains the potentiostat needed to control the biosensor and a computer to analyze the signal to generate a diagnosis of positive or negative SARS-COV-2 status with a simple user interface. The EBC collector is internally disinfected with HOCl then discarded after a single use to prevent infection of the operator.

In exemplary embodiments, the nanobody/biosensor can be implemented with new variants requiring screening and selection of a new nanobody. The nanobody/biosensor demonstrated herein detects the WA.1, alpha, beta, delta, and omicron BA.1 strains at 50 virions/ml or lower, which meets pre-determined parameters. In embodiments where a new variant arises that loses antigenicity to the current nanobody, the present disclosure enables re-testing of existing nanobody libraries and/or development of a new nanobody (or antibodies).

The disclosed breathalyzer device can improve national testing capacity as it is a fast and non-invasive technique to test queues of people waiting to enter indoor spaces, such as airports, hospitals, conference centers, or military installations, etc., where virus transmission may be prevalent. The innovations of the present disclosure include an immuno-based biosensor with a nanobody to provide specificity, as well as collection components, devices, and systems to collect aerosolized pathogens (e.g., CoV-2 viral particles) prior to testing on the biosensor.

### Nanobody-Based Electrochemical Biosensor for Real-Time Detection of Aerosolized Pathogens

In exemplary embodiments, the present disclosure describes a novel single-step method to sample exhaled breath, condense it to liquid phase and deliver it to a screen-printed electrode (SPE) that has been specially prepared for the electrochemical detection of pathogens (such as SARS-COV-2 and others, depending on the preparation method embodiment). A system embodiment of the disclosure comprises at least two units-(the EBC collection unit), and a portable potentiostat unit (PU). In exemplary embodiments, the EBC is comprised of an inclined (from about 10 degrees to about 45 degrees) superhydrophobic impaction surface, optionally the SPE, optionally a cold fluid to lower the temperature of the impaction surface, optionally at least one reservoir containing extra working fluid (or transport fluid), optionally a disinfectant, and a breathing tube. In one embodiment, the EBC includes the inclined superhydrophobic impaction surface, an electrode, a first reservoir containing a buffer, a second reservoir containing a blocking agent, and a third reservoir containing a disinfectant. The superhydrophobic impaction surface may be any suitable hydrophobic surface, including but not limited to, a film with an optional coating such as a spray coating. The buffer may be any suitable buffer, including but not limited to, a phosphate buffer, a tris buffer, or a saline buffer. The blocking agent may be any suitable blocking agent, including but not limited to, a plant or animal based albumen. The disinfectant may be any suitable disinfectant, including but not limited to, hypochlorous acid (HOCl).

In another embodiment, the EBC includes a superhydrophobic impaction surface made of polyimide treated with a spray-coating of silicone wax, a screen-printed carbon electrode, a first reservoir containing phosphate-buffered saline, a second reservoir containing bovine serum albumen, and a third reservoir containing a hypochlorous acid (HOCl) disinfectant. In exemplary embodiments, the PU includes a potentiostat, a battery, a user interface, and a microcontroller.

In the exemplary embodiments, the EBC includes a condensing chamber separated into two chambers (upper and lower) separated by the polyimide film. In the lower chamber beneath the film, a cold fluid (such as ice water or any suitable cold fluid), maintained at a temperature at least 30 degrees below that of the exhaled human breath, cools the treated polyimide. In some embodiments, the temperature of the cold fluid, the lower chamber, and/or the superhydrophobic surface is maintained from about 0° C. to about 10° C. or from about 5° C. to about 15° C., or from about 10° C. to about 20° C., or from about 15° C. to about 25° C. In the upper chamber, as breath is exhaled into the device, the gas and droplet mixture impacts the polyimide, condenses into liquid, and rolls off the inclined superhydrophobic surface into a small vial containing the SPE.

Extra working fluid (or transport fluid) is released to wash all collected breath onto the SPE. The SPE's exposed contacts are then plugged into the PU unit, a reading is conducted, and the results are reported. Once the test is concluded, the PU is disconnected and the EBC is flushed with HOCI to sterilize it. The sterilized enclosure can then be disposed of without any risk of pathogen leak/transfer to the environment.

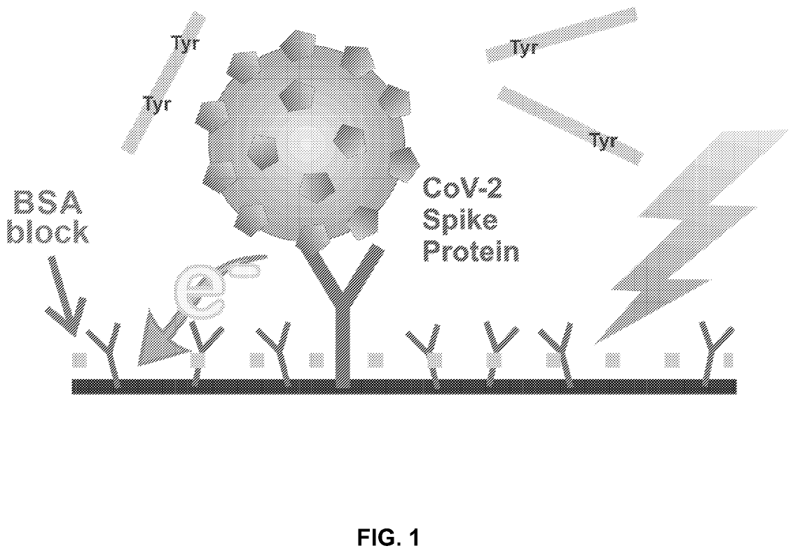

Electrochemical biosensor. Described herein is a micro-immunoelectrode (MIE) technology that uses square wave voltammetry to measure oxidation of tyrosine amino acids (at ˜0.65 V) in specific proteins (i.e., pathogen-indicating proteins). Tyrosine oxidation releases electrons that a carbon electrode detects as current (FIG. 1). The amount of current is directly proportional to the amount of analyte present. An antibody covalently attached to the electrode surface concentrates the target at the biosensor for measurement. In the case of SARS-COV-2, a nanobody (produced in llamas then sequenced and grown cost effectively in bacteria) is covalently attached to the biosensor surface to provide specificity for the biosensor. In some embodiments, one or more nanobodies are attached to the biosensor, alternative or additional to a SARS-COV-2 nanobody. In embodiments where more than one pathogen is detectable using a single biosensor, a signal (e.g., current) signal from the biosensor may be a multiplexed signal. Importantly, tyrosine oxidation is irreversible, meaning the protein bound to the nanobody on the surface of the electrode will only be measured once. This contrasts with many electrochemical sensors that measure impedance at the electrode surface; essentially measuring the binding event instead of the actual protein. The disclosed biosensor uses screen-printed, inexpensive, carbon-based electrodes (SPiCE).

Amyloid-β (Aβ) micro-immunoelectrode (MIE) of Alzheimer's disease studies. As an example of previous use of similar biosensors, the immuno-based voltametric approach was developed as the micro-immunoelectrode (MIE) biosensors to be used for minute-to-minute measures of human amyloid-β (Aβ) peptide levels in the brain of mouse models of Alzheimer's disease. The Aβ biosensors are surgically implanted into the mouse brain, enabling real-time measurement of the brain interstitial fluid in mice that are awake and freely moving. While the Aβ and SARS-COV-2 designs are different (5 μm carbon fiber pulled in glass versus a 1 mm screen printed electrode, respectively) based on their intended uses, the principle underlying the biosensors is analogous.

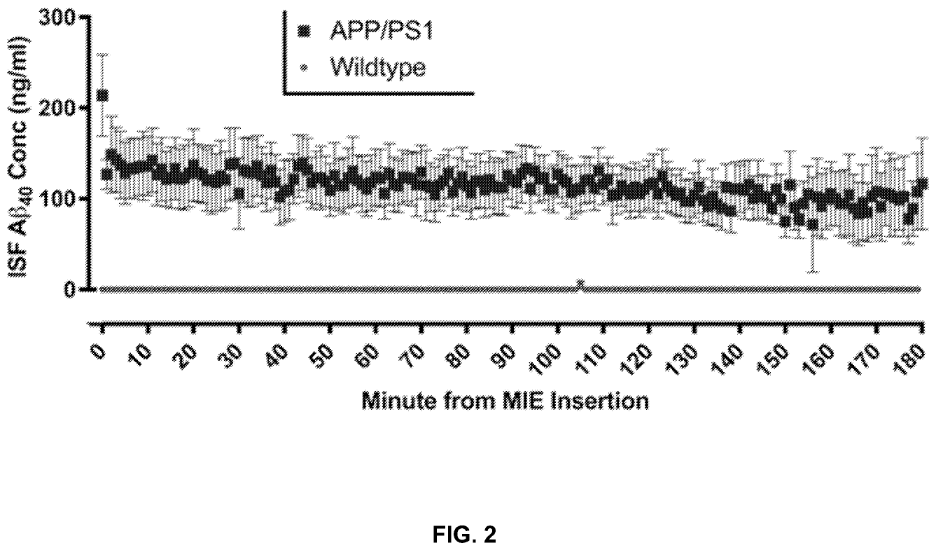

The Aβ biosensor was implanted into the brains of 1) APP/PSI transgenic mice that express human Aβ or 2) wild-type mice that only express endogenous murine Aβ. Importantly, murine Aβ lacks the tyrosine amino acid in human Aβ that, according to the theory of how the biosensors work, is required to produce the electrochemical signal on the biosensor, serving as a powerful control for specificity in vivo. In APP/PSI mice, the biosensor measured human Aβ every 60 seconds for 3 hours with minute-to-minute variability that is expected based on on-going neuronal activity (FIG. 2). In contrast, signal in the wild-type mice was negligible for the entire 3-hour measurement period. The Aβ biosensor is 8,000-fold more selective for human Aβ than any other tyrosine in the brain.

A series of biosensors were developed for use in a variety of mouse models of neurological disease, including targeting various species of Aβ peptide (Aβ₄₀, Aβ₄₂, and Aβ oligomers), tau, and α-synuclein. Another MIE was also developed against metenkephalin, a neuromodulator peptide. Standard Aβ oligomer ELISAs are generally sensitive to the low pg/ml range, whereas the biosensor is sensitive to 200 attogram/ml levels of oligomers, an approximate 10,000-fold increase in sensitivity.

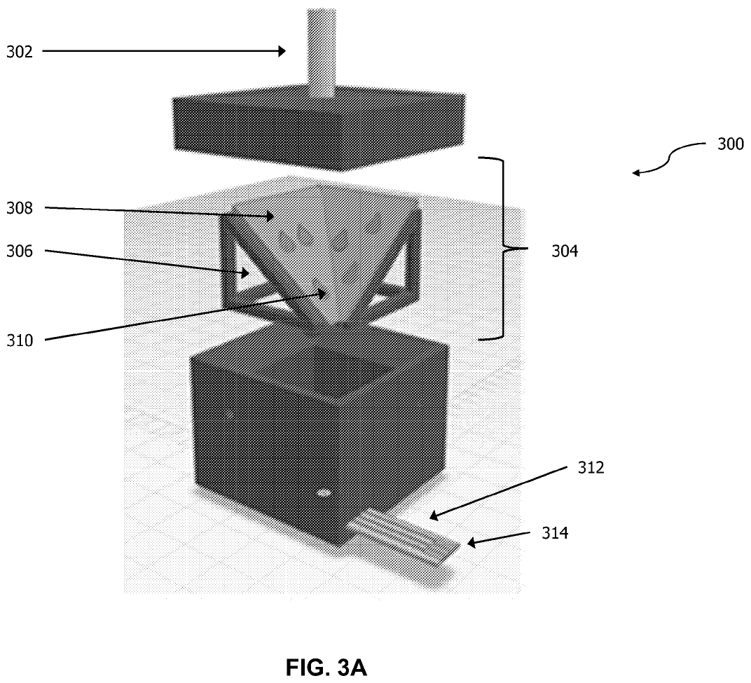

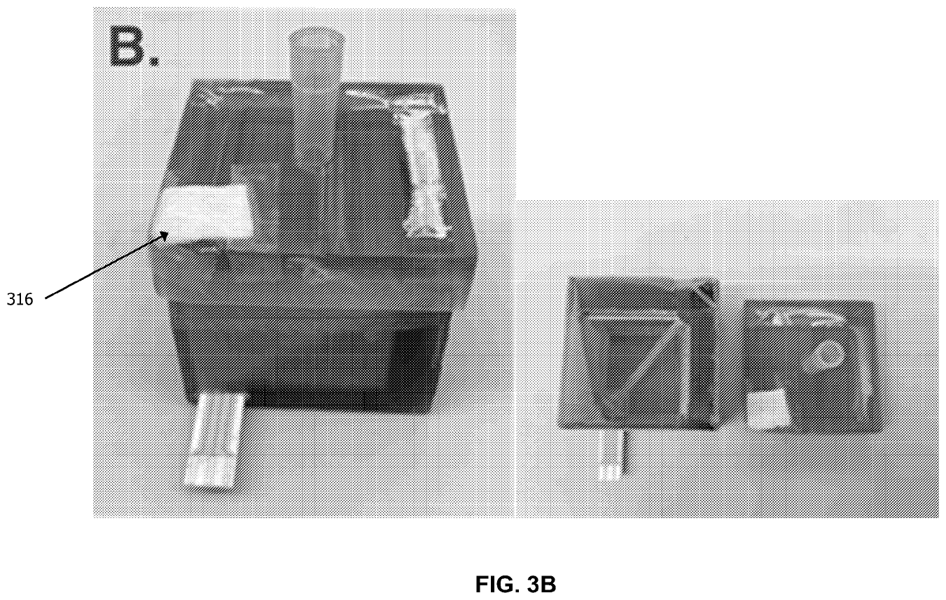

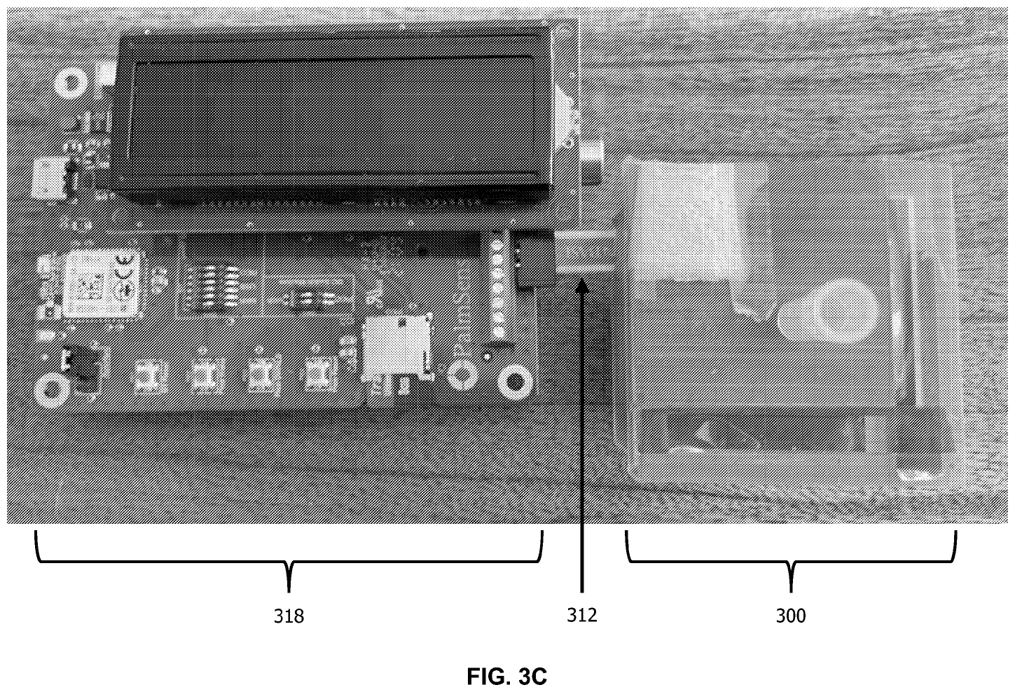

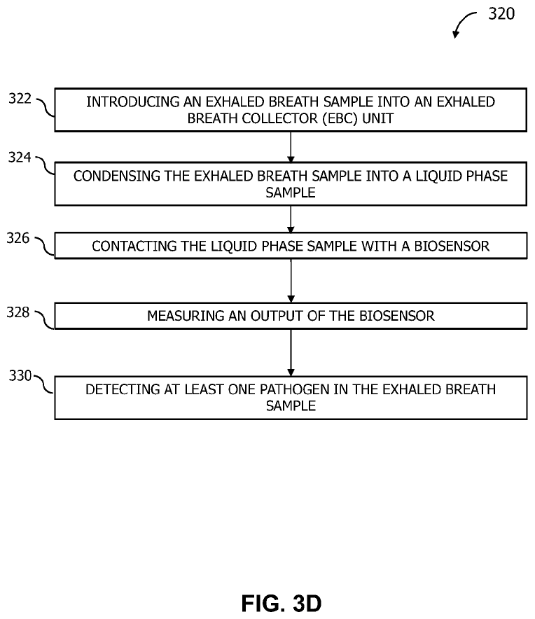

SARS-COV-2 breathalyzer. A single-use technology was developed to sample exhaled breath, condense it to liquid phase, and deliver it to the biosensor (FIG. 3(A-D)). The inside of the device includes a chilled superhydrophobic surface, such that when a warm breath contacts the cold surface, the difference in temperature causes the aerosol particles to condense on the surface. The hydrophobic layer is currently chilled with water, however the final device will be chilled by a user-initiated endothermic reaction (similar to a chemical ice pack). A user blows into the collector through a breathing tube or straw. The blowing process leads to impaction of particles on the chilled film which condenses the aerosol particles. A transport media (or working fluid) washes the aerosol droplets into a liquid phase sample for measurement upon contact with a biosensor. Depending upon the embodiment, the biosensor may be a single-use biosensor that is integrated into the single-use EBC collection device, or alternatively the biosensor may be a reusable (i.e., multi-use) biosensor that is integrated into an external analysis unit. In embodiments where the biosensor is a single-use biosensor and part of the EBC collection device, the aerosol droplets are washed onto the biosensor and the biosensor is then plugged into an external analysis unit/docking station that houses the potentiostat and software to control the biosensor and measure for SARS-COV-2. In alternative embodiments where the biosensor is an integrated part of the external analysis unit (and therefore multi-use), the liquid phase sample is suitably contacted with the biosensor and measured. After measurement, a disinfectant such as hypochlorous acid (HOCl) or any other suitable disinfectant is released into the EBC collection unit to disinfect the device. In exemplary embodiments, the entire collection device is designed to be disposable and self-disinfecting, while the analysis unit will be permanent and reusable.

FIG. 3A illustrates an exemplary EBC collection unit 300. Exhaled breath is introduced (e.g., by breathing or blowing) into EBC unit 300 via breathing tube 302. EBC unit 300 also includes a condensing chamber 304 located downstream from and in flow communication with breathing tube 302. Condensing chamber 304 includes an upper chamber (not shown) and a lower chamber 306 separated by a superhydrophobic surface 308. The upper chamber is generally defined by the space above surface 308 within condensing chamber 304. Lower chamber 306 holds a cooling fluid capable of maintaining surface 308 at a temperature conducive to condensation of exhaled breath from aerosol particles into a liquid phase 310. In some embodiments, the cooling fluid is water or a fluid produced from a user-initiated (i.e., at or immediately before the time of exhaled breath collection) endothermic reaction. In some embodiments, surface 308 is a superhydrophobic impaction surface positioned at an incline of about 10 degrees to about 45 degrees. Surface 308 may be a suitable thermoconductive film such as polyimide, with a suitable superhydrophobic coating such as a spray-coating of silicone wax.

In some embodiments, EBC unit 300 further includes a biosensor 312 located below/downstream from surface 308 such that condensed liquid phase 310 flows down onto biosensor 312. Biosensor 312 includes exposed contact(s) 314 which extend out of EBC unit 300 for connection/attachment (e.g., see FIG. 3C) to a portable potentiostat unit, a portable battery-powered electrometer,and/or an analysis unit as described herein elsewhere.

In some embodiments, EBC unit 300 optionally includes additional reservoirs (not shown) located upstream from condensing chamber 304 for introducing transport fluid for washing surface 308 and/or for introducing disinfectant for self-disinfection of EBC unit 300 prior to disposal (such as for single-use units). Transport fluid (also termed working fluid or test buffer) includes a buffer (such as phosphate buffered saline) and a blocking agent (such as bovine serum albumen), as well as other suitable transport fluids. Disinfectant includes hydrochlorous acid (HOCl) as well as other suitable disinfectants. In other embodiments, transport fluid and/or disinfectant fluid is introduced into condensing chamber 304 via port 316 (see FIG. 3B).

FIG. 3C shows EBC unit 300 with biosensor 312 communicatively coupled to a portable electrometer 318, e.g., via contact(s) located on biosensor 312 (such as exposed contacts 314 shown in FIG. 3A). In some embodiments, portable electrometer 318 may be battery powered and/or may be additionally or alternatively configured as a portable potentiostat unit. In other embodiments, biosensor 312 may be communicatively coupled to a non-portable type analysis unit.

FIG. 3D further shows an exemplary method 320 for detecting pathogens in exhaled breath according to the present disclosure. Method 320 includes introducing 322 an exhaled breath sample into an EBC collection unit, condensing 324 the exhaled breath sample into a liquid phase sample, contacting 326 the liquid phase sample with a biosensor; measuring 328 an output of the biosensor; and detecting 330 at least one pathogen in the exhaled breath sample. The exhaled breath sample is condensed on a superhydrophobic impaction surface.

Other exemplary embodiments of the present disclosure include a system for detecting pathogens, the system comprising a disposable EBC collection unit and a potentiostat unit (PU). In these embodiments, the PU comprises. a battery, a user interface, and a microcontroller, and is reusable.

In some embodiments, the method also comprises contacting the superhydrophobic impaction surface with a transport fluid to wash the condensed liquid phase sample onto the biosensor and measuring the output of the biosensor comprises connecting a potentiostat unit to the biosensor and measuring a current output of the biosensor, wherein the current output of the biosensor is based on a square wave voltammetry measurement of tyrosine oxidation of tyrosine. In some embodiments, the method further comprises flushing the EBC with a disinfectant and disposing of the EBC unit (such as for single-use units).

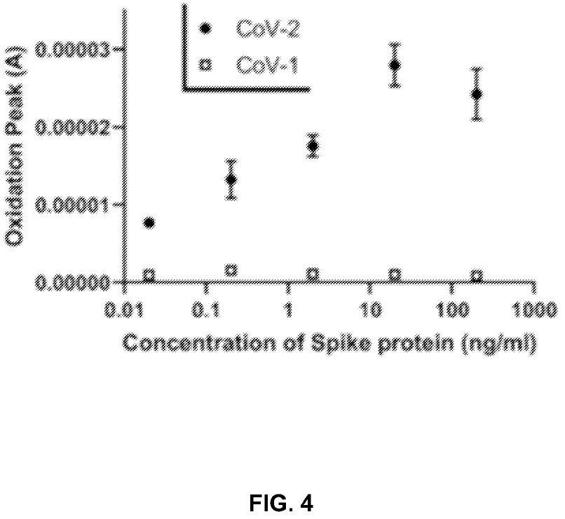

Biosensor specificity. The specificity of the CoV-2 biosensor has been tested with CoV-1 and CoV-2 spike protein with CoV-2 being detected at the low pg/mL range and negligible signal for CoV-1 (FIG. 4). The specificity of the final product is to be determined by comparing to a variety of inactivated viral particles, such as HCoV229E, OC43, HKU1, NL63, MERS, etc. Analysis shown here is supportive such that the device is expected to have each of sensitivity and specificity greater than 95%.

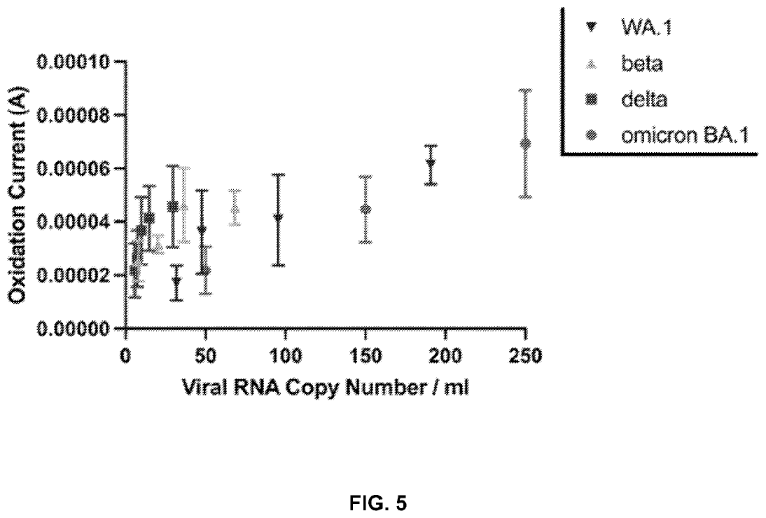

Biosensor sensitivity. The biosensor was tested with a dilution series of different variants of inactivated SARS-COV-2 (WA.1, beta, delta, and omicron BA.1) (FIG. 5). The biosensor was most sensitive for the delta and beta strains down to 10 RNA copies/ml. For all studies during development, the viral RNA copy numbers are verified using qRT-PCR. While early studies with the omicron variant demonstrate a lower limit sensitivity of 50 RNA particles/ml, a minor loss of sensitivity relative to the other variants, this is still well within the range necessary for human diagnosis. The initial tests described herein with omicron enable biosensor optimization for increased variant sensitivity. The current biosensors saturate and demonstrate a hook dose effect at high concentrations of viral particles. The length of the plateau in relation to the hook effect is still being determined for the various strains of virus.

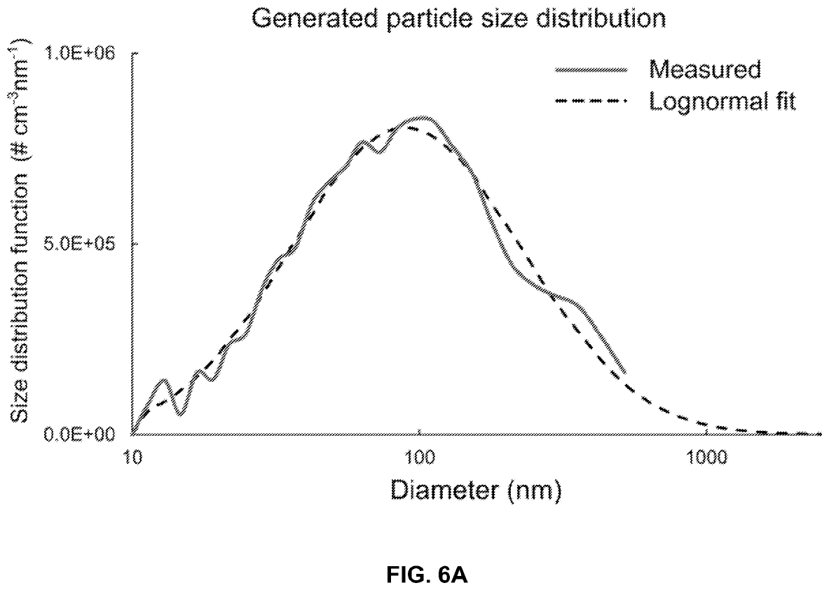

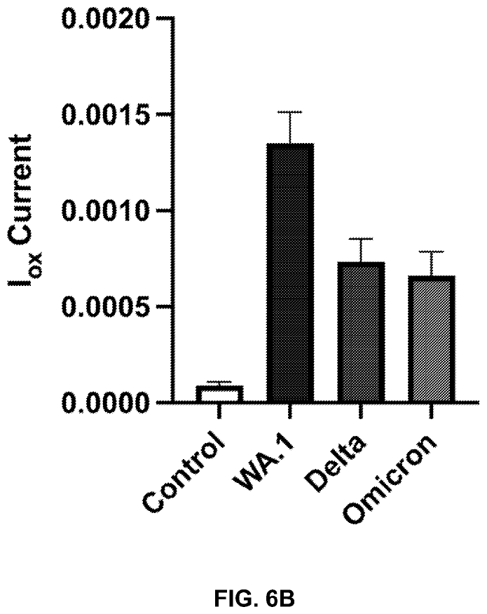

Inactivated CoV-2 particles were aerosolized then collected using the EBC collection device. The aerosolized particles had a mean peak of approximately 100 nm in diameter (FIG. 6A). The collected aerosols were manually pipetted onto the biosensor for detection. WA.1, delta, and omicron viral particles were readily detected on the biosensor compared to aerosolized control solution lacking virus (FIG. 6B).

A comprehensive analysis conducted of all prior in vitro diagnostic devices that have received an emergency use authorization (EUA) for COVID, and an understanding the current gaps in COVID testing from multiple perspectives, has helped inform device development to guide the present disclosure. Accordingly, the present disclosure enables development of the breathalyzer described herein to rapidly screen subjects entering into an enclosed space, where the risk of disease transmission is very high and/or the “cost” of disease transmission is high.

### Central Analysis Unit.

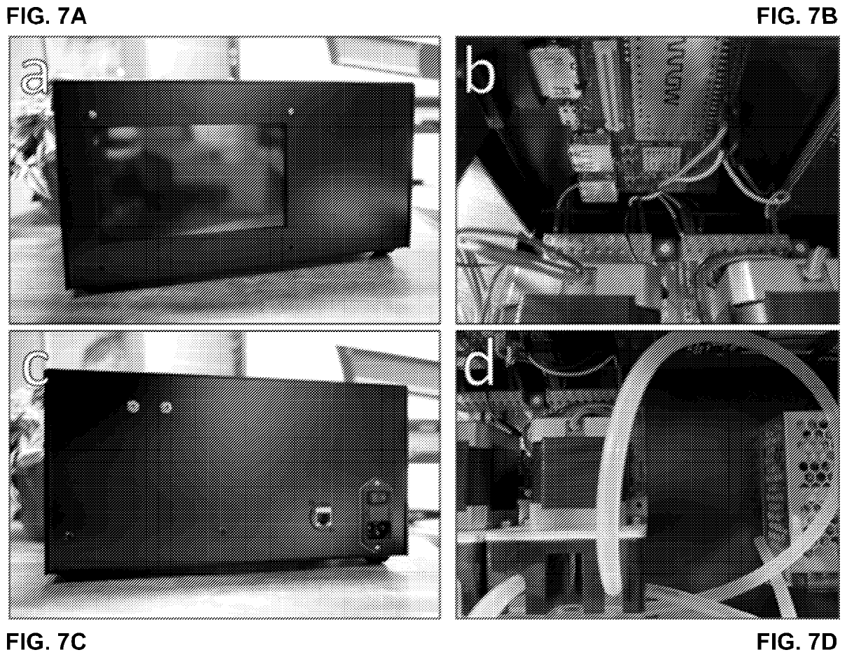

FIGS. 7A-7D show a central analysis unit containing the electrochemical sensor along with all the working fluids. This central analysis unit may be used by the environmental detector, and a similar design without the internal fluids may be used for the breathalyzer. The central analysis unit for the environmental detector has been designed such that the entire process of sample transport and analysis can be automated. This device may be user friendly such that it can be deployed to multiple locations and be operated with minimal training.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An exhaled breath condensate (EBC) collection device for collecting pathogens for analysis, the device comprising:
a breathing tube; and
a condensing chamber located downstream from and in flow communication with the breathing tube, wherein the condensing chamber includes an upper chamber and a lower chamber separated by an inclined superhydrophobic impaction surface.

2. The EBC collection device of claim 1, wherein the EBC further comprises a biosensor.

3. The EBC collection device of claim 1, wherein the inclined superhydrophobic impaction surface is positioned at an incline of about 10 degrees to about 45 degrees.

4. The EBC collection device of claim 1, wherein the inclined superhydrophobic impaction surface comprises a hydrophobic surface.

5. The EBC collection device of claim 1, wherein the lower chamber contains a cooling fluid.

6. The EBC collection device of claim 1, further comprising at least one transport fluid reservoir located upstream from and in flow communication with the condensing chamber.

7. The EBC collection device of claim 1, further comprising at least one disinfectant reservoir located upstream from and in flow communication with the condensing chamber.

8. The EBC collection device of claim 1, wherein the EBC device is disposable.

9. A method for detecting pathogens in exhaled breath, the method comprising:
introducing an exhaled breath sample into an exhaled breath condensate (EBC) collection unit;
condensing the exhaled breath sample into a liquid phase sample;
contacting the liquid phase sample with a biosensor;
measuring an output of the biosensor; and
detecting at least one pathogen in the exhaled breath sample.

10. The method of claim 9, wherein the exhaled breath sample is condensed on an inclined superhydrophobic impaction surface.

11. The method of claim 10, further comprising contacting the inclined superhydrophobic impaction surface with a transport fluid to wash the liquid phase sample onto the biosensor.

12. The method of claim 9, wherein measuring the output of the biosensor comprises connecting a potentiostat unit to the biosensor and measuring a current output of the biosensor.

13. The method of claim 12, wherein the current output of the biosensor is based on a square wave voltammetry measurement of tyrosine oxidation.

14. The method of claim 9, further comprising flushing the EBC collection unit with a disinfectant.

15. The method of claim 9, further comprising disposing of the EBC collection unit.

16. A system for detecting pathogens, the system comprising:
a disposable exhaled breath condensate (EBC) collection unit; and
a portable potentiostat unit.

17. The system of claim 16, wherein the system for detecting pathogens is used to detect viruses, bacteria, parasites, fungi, mold, or a combination thereof.

18. The system of claim 16, wherein the system for detecting pathogens is used to detect multiple pathogens in a single test.

19. The system of claim 16, wherein the system for detecting pathogens is used to detect multiple variants of a pathogen, wherein the multiple variants of a pathogen include delta and omicron variants of SARS-COV-2.

20. The system of claim 16, wherein the system for detecting pathogens is used in multiplex tests, wherein multiple pathogens are detected simultaneously.
